(19) United States
(12) Patent Application Publication
(10) Pub. No.: US 2003/0023266 A1
(43) Pub. Date: Jan. 30, 2003

(54) Individually customized atrial appendage implant device

(76) Inventors: Thomas E. Borillo, Plymouth, MN (US); Gregg S. Sutton, Maple Grove, MN (US); Jeffrey Welch, New Hope, MN (US)

Correspondence Address: FISH & NEAVE, 1251 AVENUE OF THE AMERICAS, 50TH FLOOR, NEW YORK, NY, 10020-1105, US

(21) Appl. No.: 10/200,565

(22) Filed: Jul. 19, 2002

Related U.S. Application Data
(60) Provisional application No. 60/306,557, filed on Jul. 19, 2001.

(51) Int. Cl. A61M 29/00

(52) U.S. Cl. 606/200

(57) ABSTRACT

Implant devices for modifying blood flow between an atrial appendage and its associated atrium, are customized for use in subject atrial appendages. The implant devices are tailored to uniquely match individual anatomical characteristics. Cardiac imaging techniques are used to obtain data on the size, shape and orientation of the subject atrial appendage. The raw imaging data is electronically processed using computer modeling to obtain multi-dimensional anatomical images of the subject atrial appendages. Three-dimensional computer aided design tools are used to generate customized device designs from the anatomical images of the subject atrial appendages.

This application claims the benefit of U.S. provisional application No. 60/306,557 filed Jul. 19, 2001, which is hereby incorporated by reference in its entirety herein.

### BACKGROUND OF THE INVENTION

1. Field of the Invention

The invention relates to implant devices that may be deployed in an atrial appendage. The implant devices may be used to filter or otherwise modify blood flow between the atrial appendage and an associated atrium of the heart to prevent thrombi from escaping from the atrial appendage and into the body's blood circulation system.

2. Description of the Related Art

There are a number of heart diseases (e.g., coronary artery disease, mitral valve disease) that have various adverse effects on a patient's heart. An adverse effect of certain cardiac diseases, such as mitral valve disease, is atrial (or auricular) fibrillation. Atrial fibrillation results in the loss of effective atrial contraction, and thereby altering the normal flow of blood through the atria. This often results in stasis and activation of a coagulation cascade, which leads to the formation of fibrin thrombi within the atria, and especially within the atrial appendages. The sac-like atrial appendages are frequently the source of emboli (particulates).

Blood stagnation in the atrial appendages is conducive to the formation of blood clots. The muscular ridges on the inner surfaces of atrial appendages provide convenient folds of tissue in which small thrombi (blood clots) may be trapped. These blood clots may accumulate, and build upon themselves. Small or large fragments of the blood clots may break off and propagate out from the atrial appendage into the atrium. The blood clot fragments can then enter the body's blood circulation and embolize distally into the blood stream.

Serious medical problems result from the migration of blood clot fragments from the atrial appendages into the body's blood stream. Blood from the left atrium and ventricle circulates to the heart muscle, the brain, and other body organs, supplying them with necessary oxygen and other nutrients. Emboli generated by blood clots formed in the left atrial appendage may block the arteries through which blood flows to a body organ. The blockage deprives the organ tissues of their normal blood flow and oxygen supply (ischemia), and depending on the body organ involved leads to ischemic events such as heart attacks (heart muscle ischemia) and strokes (brain tissue ischemia).

It is therefore important to find a means of preventing blood clots from forming in the atrial appendages. It is also important to find a means to prevent fragments or emboli generated by any blood clots that may have formed in the atrial appendages from propagating through the blood stream to the heart muscle, brain, or other body organs.

Some recently proposed methods of treatment are directed toward implanting a plug-type device in an atrial appendage to occlude the flow of blood therefrom.

Another treatment method for avoiding thromboembolic events (e.g., heart attacks, strokes, and other ischemic events) involves filtering out harmful emboli from the blood flowing out of atrial appendages. Co-pending and co-owned U.S. patent application Ser. No. 09/428,008, U.S. patent application Ser. No. 09/614,091, U.S. patent application Ser. No. 09/642,291, U.S. patent application Ser. No. 09/697,628, U.S. patent application Ser. No. 09/932,512, U.S. patent application Ser. No. 09/960,749, and U.S. patent application Ser. No. 10/094,730, all of which are hereby incorporated by reference in their entireties herein, describe inflatable or self-expanding devices which may be implanted in an atrial appendage to filter the blood flow therefrom.

Common catheterization methods (including transseptal procedures) may be used to implant the devices in the atrial appendages. A narrow diameter catheter delivery tube is passed through the patient's vasculature to provide a conduit or pathway to the patient's atrial appendage. The implant devices generally have an elastic or compressible structure. This structure allows a device to be compacted to a small size that is suitable for insertion in the narrow diameter catheter delivery tube. A compacted device is attached to a guide wire or a push rod, and moved through the catheter delivery tube to a deployment position within the patient's heart cavity. Then the compacted device may be expanded in situ to serve as an atrial appendage implant. The compacted devices may be of the self-expanding type (e.g., those made from shape-memory alloy materials) or may be of the type that is mechanically expanded (e.g., those that are balloon inflatable).

The success of the atrial implant treatment procedure depends on the deployment of an implant device in an appropriate position and orientation (relative to the atrial appendage). For example, for a filter device implant to be successful, the device should be positioned and oriented so that all of the atrial appendage blood flow is directed through device filter elements, and so that there is no seepage around the device. The deployed device may be retained in the appendage by engagement of the device surfaces by atrial appendage wall muscle tissue, for example, by an interference fit.

Generally, known atrial implant devices have regular geometrical shapes, for example, radially-symmetric cylindrical or oval shapes. However, the atrial appendages, though generally sac-like, have irregular geometrical shapes. Further, there may be considerable individual anatomical variation in the size and shape of atrial appendages, in addition to individual physiological variations in the nature or strength of the atrial wall muscle tissue. The use of implants having regular geometrical shapes in all cases may lead to variations in implant device treatment outcomes.

Consideration is now being given to additional atrial appendage implant device designs which take into account the anatomical and physiological variations in individual atrial appendages.

### SUMMARY OF THE INVENTION

The invention provides atrial appendage implant devices which are individually customized for use in subject atrial appendages. The implant devices are tailored to uniquely match an individual patient's physiological and anatomical characteristics.

The customized implant device may have an elastic structure of the self-expanding type or of the type that expands in an outward direction from a collapsed state to a fully expanded state using mechanical means such as a balloon or a mechanical expansion device. The self-expanding device structures may use, for example, shape-memory alloy materials or water-swellable materials such as hydrogels. The implant devices may be designed for either filtering or occlusive action on the blood flow between an atrial appendage and its atrium, and may be designed for delivery in the subject atrial appendage by either percutaneous catheterization or by surgery.

The implant device may be custom made to the specific measurements and dimensions of a subject atrial appendage. The specific measurements and dimensions of the atrial appendage may be obtained utilizing one or more diagnostic imaging methods including, but not limited to, X-ray, echocardiography, three dimensional computed tomography, and magnetic resonance imaging.

The customization process may begin with the collection of anatomical pre-operative images of the subject atrial appendage using one or more diagnostic imaging techniques. The raw imaging data may be processed using computer modeling, image synthesis, and graphics and visualization techniques to obtain a multi-dimensional image of the subject atrial appendage. The processed imaging data may be stored as a digital data file for input into suitable computer aided design (CAD) software tools. Computer aided design techniques may be used to generate three-dimensional model designs of the desired custom device. The custom device may be fabricated to the generated design specification using conventional techniques. For some device types whose fabrication involves the use of shape molds or frames, the computer aided design techniques may be used to generate three-dimensional model designs of shape molds or frames for the fabrication of the desired custom device.

Further features of the invention, its nature, and various advantages will be more apparent from the accompanying drawings and the following detailed description.

### BRIEF DESCRIPTION OF THE DRAWINGS

FIG. 1 is a partial cross-sectional view of a heart illustrating a conventional catheter entering a left atrial appendage (LAA) using a transseptal catheterization procedure.

FIG. 2 is a cross-sectional view of an exemplary left atrial appendage illustrating the unique size and shape of the individual atrial appendage.

FIG. 3 is a flow diagram illustrating several of the process steps involved in the fabrication of implant devices that are individually customized for use in an individual atrial appendage in accordance with the principles of the invention.

FIG. 4 is a schematic cross-sectional view of a preform tool made to fabricate implant devices customized for use in the atrial appendage shown in FIG. 2, in accordance with the principles of the invention.

FIG. 5 is a schematic cross-sectional view of a customized implant device fabricated using the preform tool of FIG. 4, in accordance with the principles of the invention. The implant device is of the self-expanding type fabricated from shape-memory alloy material, and is shown deployed in the atrial appendage of FIG. 2.

FIG. 6 is a schematic cross-sectional view of another customized implant device fabricated in accordance with the principles of the invention. The implant device is of the inflatable type, and is shown deployed in the atrial appendage of FIG. 2.

### DESCRIPTION OF THE PREFERRED EMBODIMENT

Implant devices for filtering or otherwise modifying blood flow between an atrial appendage and its atrium may be attached to a push rod or a shaft, and then percutaneously delivered to the appendage through a catheter delivery tube inserted in a blood vessel leading to the heart.

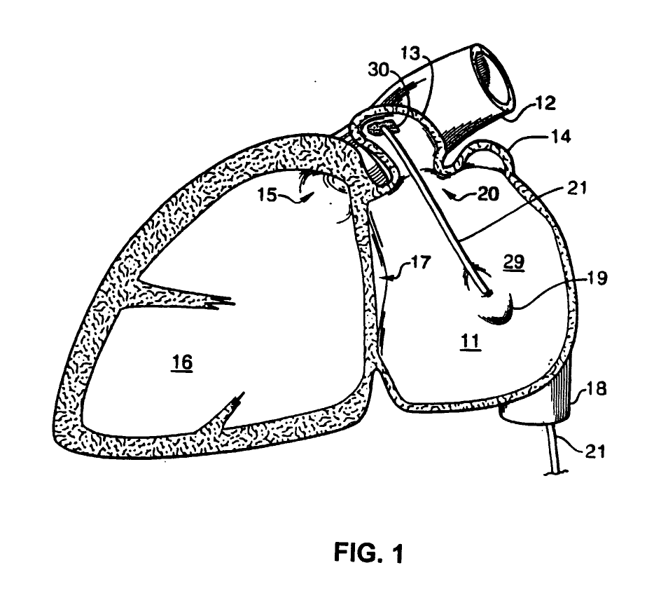

FIG. 1 illustrates, for example, catheter 21 inserted through a femoral vein (not shown) entering the right atrium of the heart through the inferior vena cava 18, and then passing into left atrium 11 through the fossa ovalis 19 or through the septum 29 before entering the left atrial appendage 13. Alternatively (not shown in FIG. 1), catheter 21 may enter the left ventricle 16 of the heart through the aorta 12, and then pass through mitral valve 17 to reach left atrial appendage 13. An implant device (not shown) attached to catheter 21 may be used to prevent thrombus 30 or emboli generated therefrom from migrating into atrium 11.

A physician's selection of the type or size of the implant device used in the implant treatment may be guided by routine pre-operative diagnostic evaluation of the heart and the atrial appendage.

Several diagnostic imaging techniques are available for clinical use. The commonly available clinical imaging techniques may be categorized by their use of either ionizing radiation or non-ionizing radiation. The techniques using ionizing radiation include techniques using X-rays (e.g., radiography, and computed tomography (CT)) or nuclear radiation (e.g., positron emission tomography). Non-ionizing radiation techniques mainly use, for example, acoustic pulses (ultrasound) for echo-ranging imaging (echocardiography) or radio-waves combined with high-field magnets (magnetic resonance imaging, (MRI)). Cardiac imaging science and technology are fields of intense research and development activity. New techniques, and improvements or refinements of older techniques are being continuously readied for clinical use. The available clinical techniques may be used to obtain planar images and also cross-sectional images (tomography) of the atrial appendage.

The inventive customization of the implant device may use one or more suitable imaging techniques or modalities, for example, computed tomography, to obtain detailed anatomical imaging data of the subject atrial appendage. The data from one or more imaging techniques or modalities may be integrated, using methods based on computer vision, image synthesis, and graphics and visualization techniques to obtain a three-dimensional image of the subject atrial appendage.

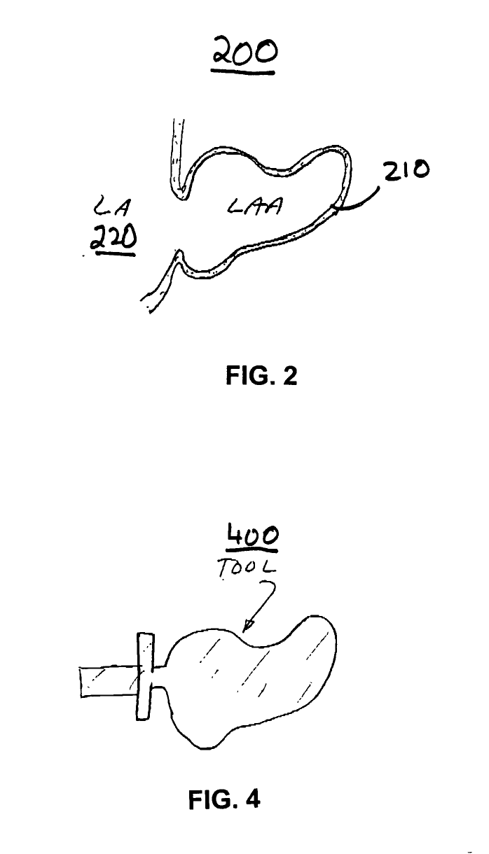

FIG. 2, for example, schematically shows, in cross-sectional view, the anatomical image 200 of a subject left atrial appendage 210. Adjoining portions of the left atrium 220 are also shown. The image provides details of the position, size and shape of atrial appendage 210. Atrial appendage 210 is seen, for example, to have a sac-like shape with an irregular diameter, and a narrow mouth (ostium).

The anatomical imaging data of the subject atrial appendage may be used to generate implant device designs which are customized for use in the subject atrial appendage, for example, by taking into account its size, shape, and orientation.

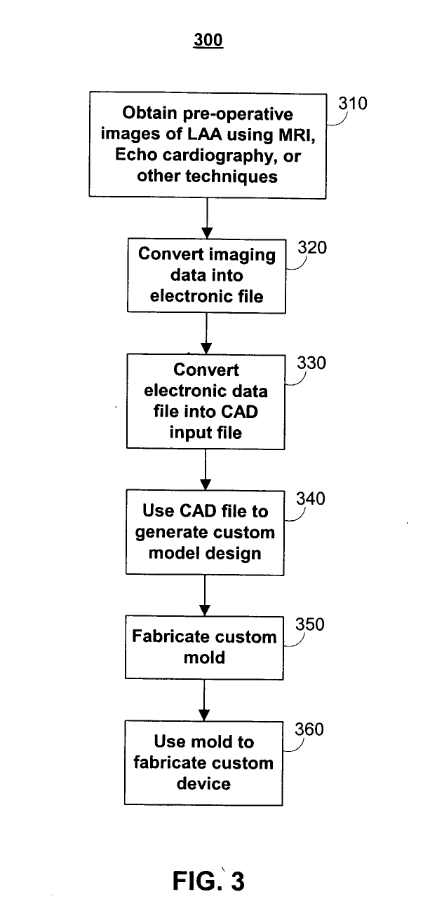

FIG. 3 shows a flow diagram of the steps that may be involved in a customization process 300, which may be used for making implant devices whose fabrication involves the use of physical frames or molds.

At step 310, pre-operative images of the subject atrial appendage are collected using one or more diagnostic imaging technique. The imaging techniques that may be used, for example, are computed tomography, echocardiography, and magnetic resonance imaging. It will be understood that the imaging techniques that may be used are not limited to the given examples. In general, any suitable imaging technique (or combination of techniques), which provides relevant anatomical information or detail, may be used. However, for ease of subsequent image data processing, three-dimensional digital imaging techniques may be naturally preferred over, for example, planar radiographic imaging techniques.

Next, at step 320, the raw imaging data collected at step 310 by one or more imaging techniques may be processed and integrated to yield an electronic representation of the subject atrial appendage anatomy. Modeling algorithms based, for example, on computer vision, image synthesis, and graphics and visualization techniques, may be used to process the raw imaging data. The algorithms may be automated, but additionally or alternatively may utilize human input. The resulting electronic representation of the subject atrial appendage anatomy may be stored, for example, as a digital data file. (FIG. 2, shows, for example, a visual image that may be created using the digital data file.)

The digital data file may have a format suitable for input into computer aided design (CAD) software tools, which for example, are commonly used for generating three-dimensional (3-D) mechanical model designs. Alternatively, at step 330 of customization process 300 the digital data file may be suitably converted or reformatted as an input data file for a suitable CAD program.

Next, at step 340 of process 300, the suitably chosen CAD software tool or program may be used to generate a model design for the custom mold or frame that may be used for fabricating the customized implant device. At step 350 of process 300, conventional machine shop techniques or methods such as machining or casting may be used to make a mold or frame according to the CAD-generated model design. The mold or frame may be made of any suitable material that is compatible with the implant device fabrication process. The suitable materials may, for example, include metals and plastics.

FIG. 4 shows, for example, a custom mold 400 according to the CAD-generated model design for fabricating implant devices that are customized for use in atrial appendage 210 (FIG. 2). Custom mold 400, as shown, has a three-dimensional solid shape, which generally conforms to the irregular geometry of atrial appendage 210.

Next, at step 360 of customization process 300, the custom implant device is fabricated using the mold or frame made at step 350. The mold or frame may be used to give a desired shape and form to the custom implant device.

A variety of filtering or occlusive implant device types may be fabricated using process 300. The implant device types that may be fabricated include the self-expanding devices, which are described, for example, in co-pending and co-owned U.S. patent application Ser. No. 09/428,008, U.S. patent application Ser. No. 09/614,091, U.S. patent application Ser. No. 09/642,291, U.S. patent application Ser. No. 09/697,628, U.S. patent application Ser. No. 09/932,512, U.S. patent application Ser. No. 09/960,749, and U.S. patent application Ser. No. 10/094,730. The self-expanding devices have elastic or compressible structures made, for example, from elastic shape-memory alloy materials. The structures are designed so that the devices may be compressed for delivery through a catheter tube. The shape-memory alloy structural materials cause the compressed devices to self expand in situ to a predetermined deployment size after they have been delivered through the catheter tube.

In the fabrication of such devices, a device preform made from shape-memory material such as nitinol may be placed over the custom mold to shape and form the implant device. The preform may, for example, be a nitinol wire mesh or suitably machined (e.g., laser cut) nitinol tube structure. Conventional heat treatment procedures may be used to give the nitinol material the desired shape-memory, which enables the device structures to self-expand to the mold shape after compression. Additional device fabrication steps may be necessary to complete the custom device fabrication. The additional steps may, for example, include attachment of blood permeable filter membranes or occlusive covers to proximal portions of the heat-treated nitinol material.

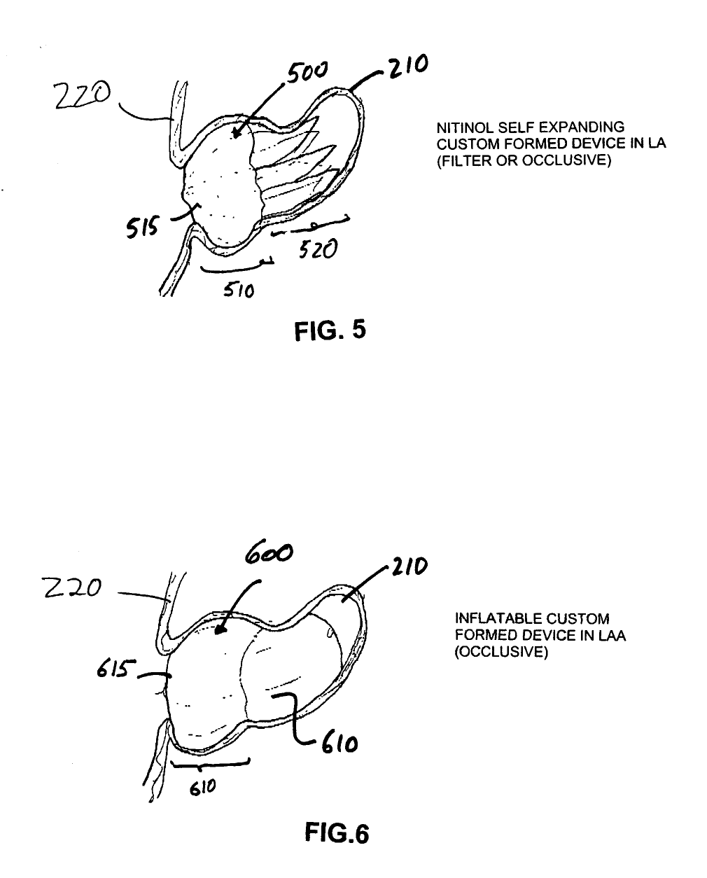

FIG. 5 shows, for example, filter implant device 500, which is customized using process 300 for use in atrial appendage 210 (FIG. 2). Implant device 500 is shown, for purposes of illustration, in an exemplary deployment position in atrial appendage 210. Deployed device 500, as shown, has a shape, which generally conforms to the irregular geometry of atrial appendage 210. Proximal cover portion 510 and distal anchor portion 520 of custom device 500 conform to and engage substantial portions of atrial appendage 210 walls. This engagement of substantial portions of the atrial appendage walls may decrease the likelihood that the deployed custom device 500 will dislodge compared to other devices that are not customized. Proximal portion 510 includes a blood-permeable membrane 515, which stretches across the ostium of appendage 210. Membrane 515 may be made of materials such as ePFTE (e.g., Gortex®), polyester (e.g., Dacron®), PTFE (e.g., Teflon®), silicone, urethane, metal fibers, or of any other suitable biocompatible material.

Optionally, an impervious membrane or cover may be substituted for blood permeable membrane 515, in which case device 500 may function as an occlusive device.

Not all atrial appendage implant device fabrication processes involve the use of shaping molds or frames. For example, device types having structures that may be expanded by mechanical means (e.g., spring biasing, or balloon inflation) may be fabricated without the use of shaping molds or frames. It will be understood that the inventive customization process may be suitably adapted for device types whose fabrication does not require or use shaping molds or frames. For example, in process 300 (FIG. 3), step 340 may be modified to generate a model design for the custom implant device directly instead of the model design for an intermediate mold or frame. The model design for the custom implant device may be used directly at device-fabrication step 360, bypassing the mold-making step 350 that was described above.

FIG. 6 shows, for example, an inflatable type implant device 600, which is customized using a modified process 300 for use in atrial appendage 210 (FIG. 2). Implant device 600 may have an inflatable plastic body 610. Implant device 600 is shown (like device 500 shown earlier in FIG. 5), for purposes of illustration, in an exemplary deployment position in atrial appendage 210. Inflated plastic body 610, as shown, has a shape, which generally conforms to the irregular geometry of atrial appendage 210. The surfaces of implant device 600 may be suitably treated to encourage tissue growth on them (so that as-implanted device 600 acquires a tissue lining). after implantation. FIG. 6 shows, for example, bio-inductive membrane 615 attached to proximal device surface portion 610. Bio-inductive membrane 615 may, for example, be a polymer membrane, which has been treated with biochemical agents that promote endothelial cell attachment.

Other examples of self-expanding implant devices that may be fabricated using the inventive customization process are those made from water-swellable material. The water-swellable material may be any suitable water absorbing resin, epoxy, or polymeric material. These materials may, for example, be cross-linked copolymers such as those based on polyethylene glycol, polyvinyl alcohol, poly acrylamide, and polyvinyl pyrrolidone, or other water-absorbing polymers that are commonly referred to as hydrogels. The water-swellable material absorbs water, and swells when placed in contact with blood. The dry water-swellable material may be formed (e.g., according to the device design generated at step 340, FIG. 3) so that it's swollen-state shape conforms to the shape of the subject atrial appendage.

It will be understood that the foregoing is only illustrative of the principles of the invention, and that various modifications can be made by those skilled in the art without departing from the scope and spirit of the invention. For example, the implant device types can differ from the specific examples mentioned herein, and the inventive customization method may be used for implant devices for other body cavities other than the atrial appendages mentioned herein.

1. A method for customizing an implant device for use in an atrial appendage comprising:
collecting anatomical data on said atrial appendage;
generating a model device design from said anatomical data; and
fabricating a customized implant device according to said model device design.

2. The method of claim 1 wherein said collecting anatomical data comprises collecting multi-dimensional data.

3. The method of claim 1 wherein said collecting anatomical data comprises using cardiac imaging techniques to collect raw data.

4. The method of claim 3 wherein said using cardiac imaging techniques comprises using a technique selected from the group of computed tomography, magnetic resonance imaging, and echocardiography.

5. The method of claim 1 wherein generating a model device design from said anatomical data further comprises using a computer aided design software tool to generate said model design.

6. The method of claim 1 wherein generating a model device design from said anatomical data further comprises processing said anatomical data to generate a multi-dimensional image data file.

7. The method of claim 6 wherein generating a model device design from said anatomical data further comprises using a computer aided design software tool to generate said model design from said multi-dimensional image data file.

8. The method of claim 1 wherein fabricating a customized implant device according to said model device design further comprises shaping an inflatable structure.

9. The method of claim 1 wherein fabricating a customized implant device according to said model device design further comprises shaping a spring-biasable structure.

10. The method of claim 1 wherein fabricating a customized implant device according to said model device design further comprises shaping a self-expanding structure.

11. A method for fabricating a custom implant device for use in an atrial appendage comprising:
collecting anatomical data on said atrial appendage;
generating a model design from said anatomical data;
fabricating a shape mold according to said model design; and
fabricating a customized implant device using said shape mold.

12. The method of claim 11 wherein said collecting anatomical data comprises collecting multi-dimensional data.

13. The method of claim 12 wherein said collecting anatomical data comprises using cardiac imaging techniques to collect raw data.

14. The method of claim 13 wherein said using cardiac imaging techniques comprises using a technique selected from the group of computed tomography, magnetic resonance imaging, and echocardiography.

15. The method of claim 12 wherein generating a model design from said anatomical data further comprises using a computer aided design software tool to generate said model design.

16. The method of claim 12 wherein generating a model device design for a shape mold from said anatomical data further comprises processing said anatomical data to generate a multi-dimensional image data file.

17. The method of claim 16 wherein generating a model device design for a shape mold from said anatomical data further comprises using a computer aided design software tool to generate said model design from said multi-dimensional image data file.

18. The method of claim 12 wherein fabricating said shape mold according to said model design further comprises shaping a solid body

19. The method of claim 12 wherein fabricating a customized implant device using said shape mold further comprise placing shape-memory alloy material on said shape mold.

20. The method of claim 19 wherein said shape-memory alloy material comprises nitinol.

21. The method of claim 19 wherein fabricating a customized implant device using said shape mold further comprises heat treating said shape-memory alloy material.

22. The method of claim 19 wherein fabricating a customized implant device using said shape mold further comprises attaching a blood-permeable membrane to said shape-memory alloy material.

23. The method of claim 19 wherein fabricating a customized implant device using said shape mold further comprises attaching a blood impervious membrane to said shape-memory alloy material.

24. A device for modifying blood flow through the ostium of an atrial appendage, wherein the appendage has an irregular geometric shape, comprising:
a body; and
a cover disposed on said body, wherein said cover extends across said ostium, and wherein said body has a shape substantially conforming to said irregular shape of said atrial appendage.

25. The device of claim 24 wherein said body comprises an inflatable structure.

26. The device of claim 24 wherein said body comprises a self-expanding structure.

27. The device of claim 26 wherein a self-expanding structure comprises shape-memory alloy material.

28. The device of claim 27 wherein said shape-memory alloy material comprises a wire mesh.

29. The device of claim 28 wherein said shape-memory alloy material comprises a machined tube structure.

30. The device of claim 24 wherein said body comprises a structure that has been formed using a mold having a shape substantially conforming to said irregular shape of said atrial appendage.

31. The device of claim 24 wherein said cover comprises a filter membrane.

32. The device of claim 24 wherein said cover comprises a blood impervious membrane.

33. An implant device for modifying blood flow through the ostium of an atrial appendage, wherein the appendage has an irregular geometric shape, comprising a water-swellable material body, wherein said body comprises a proximal portion and a distal portion, and wherein said body has a dry shape and a swollen shape.

34. The implant device of claim 33 wherein said swollen shape substantially conforms to said irregular geometric shape of said atrial appendage.

35. The implant device of claim 33 wherein said body dry shape is formed such that on absorbing water said swollen shape substantially conforms to said irregular geometric shape of said atrial appendage

36. The implant device of claim 33 wherein said water-swellable material comprises hydrogels.

37. The implant device of claim 33 wherein said water-swellable material comprises cross linked copolymers.

38. The implant device of claim 37 wherein said cross linked copolymers are based on polymers selected from the group consisting of polyethylene glycol, polyvinyl alcohol, poly acrylamide, and polyvinyl pyrrolidone.
